(19) United States
(12) Patent Application Publication
(10) Pub. No.: US 2020/0060682 A1
(43) Pub. Date: Feb. 27, 2020

(54) MEDICAL DEVICE FOR PLACING A CATHETER

(71) Applicant: Eberhard Karls Universitaet Tuebingen Medizinische Fakultaet, Tuebingen (DE)

(72) Inventor: Bianca Haase, Tuebingen (DE)

(21) Appl. No.: 16/673,695

(22) Filed: Nov. 4, 2019

Related U.S. Application Data
(63) Continuation of application No. PCT/EP2018/061484, filed on May 4, 2018.

(30) Foreign Application Priority Data
May 5, 2017 (DE) 10 2017 109 723.6

(51) Int. Cl. A61B 17/122; A61M 25/01

(52) U.S. Cl. CPC A61B 17/122; A61M 2025/0681; A61M 25/0111

(57) ABSTRACT

The disclosure relates to a medical device for placing a catheter in an umbilical cord blood vessel of a newborn, wherein the medical device comprises a longitudinal axis, a proximal and a distal end, the medical device being designed as a sheath to enclose a portion of an umbilical cord. The medical device comprises a first unit comprising a clamping device for clamping the umbilical cord, and/or a second unit, comprising a severing device for severing the umbilical cord to form an umbilical cut surface, and a third unit comprising a hooking device for spreading the umbilical cut surface.

### CROSS-REFERENCE TO RELATED APPLICATIONS

This application is a continuation application of International patent application PCT/EP2018/061484, filed May 4, 2018, which international patent application claims priority to German patent application DE 10 2017 109 723.6, filed May 5, 2017. The entire contents of these priority applications are incorporated herein by reference.

### BACKGROUND

The disclosure relates to a medical device for placing a catheter in an umbilical cord blood vessel of a newborn.

During the initial care of a newborn or preterm infant, access may be required to administer medication to the newborn. It is particularly important that the initial care of the newborn is particularly fast and securely in a resuscitation setting.

In particular, three different options are known in the prior art, which are regularly used: the umbilical catheter, the peripheral vein access and the intraosseous access (IOS). All access are provided by experienced personnel. This requires a lot of practice and experience on the part of the treating persons. Especially with the first mentioned alternative, several people are busy laying the access. This often leads to space problems around the newborn, since with the care of the newborn still further persons are tasked with the care of the newborn.

The rapid placement of a peripheral venous catheter (PVK) is carried out by specially trained and experienced personnel. However, this does not always succeed, and the more immature and hypotonic the newborn is, the more difficult it is. Particularly during resuscitation, the placement of the catheter is often particularly difficult. The back of the hand, the back of the foot, the head veins and the cubital veins can be used as puncture sites. Especially in very small preterm infants and newborns with poor circulation, peripheral venous access is usually not possible.

As an alternative access option, an IOS access can be applied over the proximal tibia. However, the IOS is only permissible from a weight of 3000 grams and bears additional risks, such as bone injuries, inflammation and growth disorders of the lower led bone. At present, however, it is technically easier to place an IOS-access compared to an umbilical catheter and is therefore much faster.

A further alternative access option is the umbilical catheter. Here, the blood vessel is punctured or cannulated with the catheter. The blood vessel can be an umbilical vein or an umbilical artery. A venous or arterial access in the first few minutes of life is essential to administer volume boluses, glucose and emergency medications such as suprarenin to critical ill newborns. If this is not successful, consequential damage such as hypertension, hyperglycemia or prolonged bradycardia is likely to occur.

The biggest problem when placing an umbilical vein catheter (NVK) or umbilical artery catheter (NAK) is to detect the blood vessels and prepare them so that the lumen becomes visible. Currently, this is achieved by manually spreading of the umbilicus with two surgical forceps. This requires at least one person to present the umbilicus such that a second person can place the catheter.

Thus, there are two major disadvantages when placing an umbilical catheter. On the one hand, it requires at least two experienced physicians, who are coordinated to place a catheter. These physicians both work actively on the umbilicus, so that space problems can occur around the newborn. On the other hand, it requires a lot of experience of the treating physicians. Placing a catheter is usually very difficult and increasingly frustrating.

### SUMMARY

According to an aspect of the disclosure, it is an object disclosure to provide for a novel medical device for placing a catheter in an umbilical cord blood vessel, which simplifies the difficult insertion of a catheter via an umbilical cord blood vessel.

According to an aspect of the disclosure, the object is solved by a medical device for placing a catheter in an umbilical cord blood vessel of a newborn or preterm baby, wherein the medical device comprises a longitudinal axis, a proximal and a distal end, and being formed as a sheath to enclose a portion of an umbilical cord, wherein the device comprises the following: a first unit, comprising a clamping device for clamping the umbilical cord, and/or a second unit, comprising a severing/cutting device for severing/cutting the umbilical cord to form an umbilical cut surface, and a third unit, comprising a hooking device for spreading the umbilical cut surface.

According to another aspect of the disclosure, the object is achieved by a use of the medical device for placing a catheter in an umbilical cord blood vessel of a newborn, in particular an umbilical vein or umbilical artery catheter.

Finally to another aspect of the disclosure, the object of the disclosure is also achieved by a method for placing a catheter in an umbilical cord blood vessel of a newborn using the medical device, wherein the method comprises the following successive steps:
applying the medical device around the umbilical cord of a newborn;
actuating the first unit to clamp the blood vessels of the umbilical cord at the proximal end of the device;
actuating the second unit, comprising the severing device, to cut the umbilical cord and to form an umbilical cut surface;
removing the second unit from the device;
actuating the third unit to spread the umbilical cut surface; and
placing the catheter in an umbilical cord blood vessel.

According to a preferred embodiment of the method according to the disclosure, after the last step of placing the catheter in an umbilical cord blood vessel a further step can be provided:
detaching the third unit and hooking the placed catheter into an appliance of the first unit which is intended for this purpose.

This also can completely solve the problem.

Advantageously, with the medical device according to the disclosure, one treating person quickly can place a catheter in an umbilical cord blood vessel of a newborn securely and fast. This is achieved by the advantageous design of the medical device, which is able to clamp and to sever/cut an umbilicus and to spread an umbilical cut surface. The blood vessels of the umbilicus are presented in a particularly visible way. Thus, the medical device enables the safe placement of the catheter.

Furthermore, the medical device offers the advantage that a fast and safe access can be accomplished in all weight classes of newborns and preterm infants. This is achieved by variable size of the device, so that the medical device can be used for both very small preterm infants and normal-weight newborns.

According to the disclosure, “newborn” may also be a preterm infant, especially a preterm infant weighing less than 3000 grams. The newborn is in particular a human or a mammal.

According to the disclosure, the term “umbilicus” is used as a synonym for “umbilical cord”. The umbilical cord blood vessels are on the one hand the umbilical vein and on the other hand the two umbilical arteries. A catheter can be placed in both in the umbilical artery and in the umbilical vein.

In order to be able to place the catheter, first the medical device is placed around the umbilicus, such that a part of the umbilicus cord is enclosed by it. The device can be placed and closed around the umbilicus or the umbilicus can be threaded into the device. The device can be designed such that it is available in different diameters and can be selected to suit the respective umbilicus diameter.

Preferably the medical device is placed around the umbilicus such that the proximal end of the device is as close as possible to the body of the newborn and the distal end of the device is as far away as possible from the body of the newborn.

The first unit of the medical device is designed such that it comprises a clamping device. Wth the clamping device the umbilical cord is clamped, especially the blood vessels. Furthermore, the first unit is designed such that the clamping device can be opened and closed reversible. On the one hand, the blood vessels should be securely clamped so that bleeding of the newborn is prevented. On the other hand, however, when the catheter is placed, the blood vessels should be able to be opened, so that the catheter can be guided properly and then closed again to fix the placed catheter in place. For this purpose, the first unit comprises a clamping device, which can be opened and closed reversibly.

According to the disclosure, the medical device may comprise the first unit and the second unit in combination with the third unit, or may comprise the first unit or the second unit each in combination with the third unit, optionally with either the second unit or the first unit.

According to an aspect of the disclosure, the first unit comprises fastening elements, which allow the catheter to be fastened. For example, the catheter can be clamped to the outside of the first unit. Thus, a displacement of the placed catheter is prevented with the first unit as it fixes the catheter.

The second unit of the medical device is designed such that it comprises a severing device. With the severing device the umbilical cord is severed to obtain an umbilical cut surface. Preferably, the umbilical cut surface is cut as smooth as possible, such that the tissue is not frayed. This facilitates the identification of the blood vessels and the distinction between umbilical vein and umbilical artery. The severing device can comprise any form of a cutting means, with which tissue can be cut. In addition, the second unit can comprise elements that particularly simplify cutting to handle.

The third unit of the medical device is designed such that it comprises a hooking device. With the hooking device it is possible to spread the umbilical cut surface and to present the blood vessels of the umbilicus. Thus, the umbilical vein and the two umbilical arteries are clearly presented, so that it is easy for a treating physician to place a catheter in one of the umbilical cord blood vessels. The hooking device is designed, such, that it comprises elements which are suitable for hooking into the umbilical tissue. The elements are moved by a movement sequence, such, that the hooked tissue is presented.

In order to be able to place a catheter with the medical device according to the disclosure, the medical device is first applied around the umbilical cord of the newborn. Because of a movement mechanism within the first unit, the clamping device is actuated, such, that the umbilicus is clamped, preferably at the proximal end. For example, the clamping can be enabled by elements that bind, pinch, squeeze or clamp the umbilicus. In particular, the blood vessels are clamped without rupturing the tissue.

In a next step, a movement mechanism activates the severing device within the second unit, such that the umbilicus is severed. Thus, the second unit is separated/detached together with the umbilical end. Then, the umbilicus is only surrounded by the first and the second unit. Then, the newborn only has a short portion of the umbilicus, which comprises an umbilical cut surface at the distal end.

In a next step, the hooking device of the third unit is also activated by a movement mechanism. Here the umbilicus is spread, such that the umbilical vessels are clearly presented.

The movement mechanism, which actuates the clamping-, the severing- and the hooking device can be activated by a rotation, shifting, pressing against each other etc. Preferably, the movement mechanism is configured such that a physician can activate the mechanism on his own, i.e. a maximum of two hands are required. The movement mechanism of the individual units can be activated one after another such that the clamping-, the severing- and the hooking devices are activated individually. Furthermore, a single movement mechanism can also activate the respective clamping-, severing- and hooking device simultaneously in a kind of chain reaction. For example, the rotation of different units against each other can trigger the mechanism of several units. For this, the units are interconnected such that a simultaneous activation of the movement mechanism can take place.

According to a preferred refinement, the first unit is arranged at the proximal end of the device and the second unit is arranged at the distal end of the device, wherein the third unit is arranged between the first and the second unit of the device.

This refinement offers the advantage that it ensures that the umbilicus is cut at a preferred point, that the blood vessels of the umbilicus are severed at a preferred point and that the umbilical cut surface is spread at a preferred point.

This refinement also offers the advantage that the individual units are synchronized such that the umbilical cord is clamped at the proximal end of the device, i.e. as close as possible to the newborn, while the umbilical cord is cut at the distal end of the device, i.e. away from the newborn. Advantageously, the third unit is arranged between the second and the first unit, as the umbilical cord must first be cut to spread the umbilical cut surface.

According to another refinement, the first, second and third unit are connectable to one another via connecting means, such, that the first unit is attachable in the device by rotation against the second and/or third unit, wherein the connecting means are preferably selected from a rotational connection, bayonet connection, rail connection, hook connection and/or magnet connection.

According to a refinement, the three units are initially connected to each other. This offers the advantage that the device is enclosed around the umbilicus all at once, and that each unit does not have to be placed or threaded individually around the umbilicus. This makes is easier to apply the medical device and thus also to place a catheter. This also ensures that there is no confusion between the individual units when the device is applied.

Furthermore, this refinement offers the advantage that the connecting elements can be made re-closable so that the individual units can be connected and disconnected from each other. Especially, in the method for placing a catheter it is advantageous, that the individual units can detach from each other. Thus, the first unit can be removed together with the umbilical end after the umbilicus has been severed. The third unit can preferably be detached from the first unit after the catheter has been placed, while the first unit preferably remains at the umbilicus, thus preventing the catheter from displacing.

The first unit can also act as an umbilicus clamp. This can therefore remain on the newborn for longer, even if the catheter has already been pulled out.

According to another preferred refinement, the sheath is closed for threading the umbilical cord into the sheath.

According to this refinement, the individual units can be reversibly connected to each other, while the individual units are closed in the longitudinal direction. In order for the device to enclose the umbilicus, the umbilicus is threaded into the device.

This refinement offers the advantage that, due to the closed sheath, it is ensured that the individual units cannot open during the method of placing a catheter. For example, an opening of the first unit could lead to bleeding of the newborn. In addition, it can be prevented that the umbilicus is hooked or squeezed in the device.

According to a preferred refinement the sheath can be opened or slid open or pushed open along the longitudinal axis of the medical device and can be locked again to close the sheath for applying the device around a portion of the umbilical.

The longitudinal opening and re-closing of the sheath makes it easy to place the medical device around the umbilicus. Thus, the medical device can be applied around the umbilicus and closed with a simple handle. In particular, it is closed in such a way as to prevent undesirable opening of the device.

This refinement enables an opening and re-closing of the individual units. Thus, the individual units can be removed from the umbilicus if required. For example, after the catheter has been placed, a simple opening or sliding open can remove the third unit.

According to this refinement the appliance of the medical device around a section of the umbilical cord is simplified, as threading the umbilical cord is not required, which can be very time-consuming if there is already an umbilical clamp on the umbilicus. Thus, this refinement offers a time saving.

According to a preferred refinement of the medical device the first, second and/or third units each comprises sheath closing means for opening or sliding open the sheath, wherein the sheath closing means are selected from clamp, click, hinge, hook and/or rail means.

With these sheath closing means it can be ensured that the respective units can be opened and closed reversibly. The sheath closing means are designed such that after the medical device has been enclosed around the umbilicus, none of the units is opened at an undesirable time. Depending on the design of the medical device, a clamp, click, hinge or rail means may be preferred as a sheath closing means.

According to another preferred refinement of the medical device according to the disclosure the clamping device comprises a tie or an air cushion for clamping blood vessels in the umbilical cord at the proximal end of the medical device.

By using a tie, it is possible to clamp the umbilical cord securely and open it reversibly, if necessary. In this case, the umbilicus is not ruptured in a detrimental way. For example, the tie can be attached in the first unit via a loop guide such that the umbilicus can be clamped by pulling the tie. The ribbon then remains in this position unless the tie is loosened again by a movement mechanism. In addition, the tie can also be tightened or loosened by another movement mechanism.

The same can be achieved with an air cushion, which can be present in the first unit as a clamping device. This is filled with air or another gas as required, such that the blood vessels are clamped. Furthermore, the air is released from the air cushion if the opening of the blood vessels is necessary, for example when placing the catheter. By using the air cushion, a particularly gentle clamping of the umbilicus is possible, since a rupture of the umbilicus can be nearly excluded here.

According to another preferred refinement of the medical device according to the disclosure the clamping device is designed, such, that the umbilical cord is clamped by equatorial rotation of the first unit against the second and/or third unit.

By this simple mechanism, through the opposite rotation of the individual units, it is quickly possible for a treating physician to clamp the blood vessels. This eliminates the need of further manual handling by the treating physician.

Alternatively, the first unit can also be designed to be detachable from the medical device such that it can be detached during use if necessary. Wth the first unit, clamping can be achieved by a rotary movement, which reduces the lumen and finally allows the umbilical cord to be clamped. Alternatively, the lumen of the first unit may be reduced by pulling one or two tie-/cable-like element/elements provided on the first unit. The tie-/cable-like elements can be elastic or inflexible, and can be made of rubber, plastic, textile, etc., or mixtures thereof. For example, a cord stopper element can be provided for tightening and fixing.

According to another preferred refinement of the medical device the severing device as well as the hooking device are activated by rotating the second unit against the third unit or against the first unit.

This refinement provides the advantage, that the umbilical cord is severed and the umbilical cut surface is spread out immediately afterwards, triggered by only one movement. This leads to a time saving when placing a catheter. According to this refinement, the third unit is preferably designed, such, that it comprises, for example, a spring device at the hooking device. This spring device can be tensioned if the severing device of the second unit has been activated by rotating. Once the umbilicus has been severed, the tensioned spring device can relax such that the third unit's hooking device is activated to spread the umbilical cut surface.

According to another preferred refinement of the medical device according to the disclosure the severing device comprises cutting means preferably selected from a knife-like cutting means, a scissor-like cutting means or a thread-like cutting means.

According to this refinement, the umbilicus can be cut by the cutting means. Preferably, the cutting means are able to provide a particularly smooth umbilical cut surface such that the blood vessels are clearly identifiable.

Preferably, the knife-like cutting mean is a sharp-edged metal, in particular a scalpel blade or knife blade.

The scissor-like cutting means can be one, two or three or four or five knife-like cutting means, which cut off the umbilical cord end using a scissor-like mechanism. Preferably, the umbilicus is not squeezed, but cut as smooth as possible.

The thread-like cutting mean according to the disclosure can be a metal thread, which is able to cut tissue particularly smoothly. In addition, here the severing of the umbilicus takes place preferentially without the umbilicus being squeezed.

Preferably, the cutting means are designed, such, that the treating physician can operate this cutting means securely with one hand, so that he does not require the help of another person. Preferably, the cutting means comprise user protection that prevents the user from being injured by the blades.

According to another preferred refinement of the disclosure the knife-like cutting means are movably attached to a rail or the knife-like cutting means are movably attached to one or more hinges, or the knife-like cutting means are spring-loaded.

This refinement has the advantage that the umbilicus can be severed using different cutting mechanisms. The cutting along a rail enables smooth cutting of the umbilicus, as the cutting means is guided along the umbilicus. The cutting by a rail guide ensures that the cut is particularly straight or smooth.

If the cutting mean is movably attached to a hinge, the severing of the umbilicus can be carried out in a similar way as cutting with a guillotine. Since the cutting means is attached to a hinge, this also ensures particularly smooth cutting of the umbilicus, as the cutting mean is guided by a hinge. For example, the knife-like cutting means can be designed similar to a pair of scissors.

If the cutting means is spring-loaded, the cutting can be carried out similar to cutting with scissors, whereby the cutting means returns to its original position after the cutting. According to this refinement, the knife-like cutting means is preferably designed with two knifes.

According to the disclosure, any cutting means and any cutting mechanism can be used which are known in the state of the art and is suitable for severing through tissue. For example, the cutting means can be designed like a cigar cutter.

Furthermore, the cutting means is designed, such, that the cutting mechanism, i.e. the cutting itself, is easy to handle. Preference is given to cutting means, which can be arranged in the second unit in a particularly space-saving manner. Preferably, the cutting knife is made of stainless steel with a sharpened cutting edge.

According to another refinement of the medical device according to the disclosure, the severing device of the second unit is designed, such, that the umbilical cord is clamped by equatorial rotation of the second unit against the first and/or third unit, and wherein the second unit is detachable arranged on the device.

This refinement has the advantage that the treating physician can sever the umbilical cord by simply rotating the second unit. According to this refinement the severing device can be designed such that, by rotating the second unit, a mechanism is activated such that a cutting means severs the umbilicus. By simultaneously releasing the second unit, after rotating and cutting the umbilicus, the second unit with the remaining umbilicus is severed/separated/detached from the newborn. This has the advantage that only the first and third units are present at the umbilicus of the newborn. In a next step, the third unit can spread out the umbilical cord cut and thus present the veins or arteries.

According to another refinement of the medical device according to the disclosure, the hooking device comprises at least three hook elements, preferably comprises at least four, five, six, seven, eight, nine or ten hook elements each having a first fastening end and a second hooking end.

The first fastening ends of the hook elements is preferably attached to a hinge within the third unit such that the hook elements can move in one direction if necessary. Preferably, the hook elements move in the longitudinal direction.

The other end of the hook elements, namely the first hooking end, is preferably designed such that it can penetrate into the umbilical tissue and can be hooked into it. In a preferred refinement, the first hooking end can comprise barbs, which prevent the umbilical tissue from being detached from the hook again.

Due to the number of hook elements, it can be ensured that the umbilical cut surface is particularly advantageously spread out. The number of hook elements depends primarily on the diameter of the medical device. A medical device for a smaller umbilicus can be designed to comprise a smaller diameter. This device preferably comprises fewer hook elements than one for a larger umbilicus.

Instead of hooks, device can be provided that sucks in the umbilicus by negative pressure at the upper edge, or that pinches the upper edge from the outside and then spreads it out.

According to another refinement of the medical device according to the disclosure the hooking device comprises hooking elements and a ring element, wherein the hooking elements are each attached to the ring element via a first fastening end, which is attached to the hooking device circumferentially in hinges around the sheath, and wherein a second hooking end of the hooking elements is designed for hooking into the umbilical cord tissue.

This refinement provides the advantage that the hook elements are all attached to one ring element and can therefore all be moved simultaneously.

According to another refinement of the medical device according to the disclosure the ring element is—in distal and proximal longitudinal direction of the medical device—slidably arranged on the hooking device such that by moving the ring element from a distal position in a proximal position the hook elements can hook in the umbilical cord and can spread the umbilical cut surface.

With this refinement, it is possible for a treating physician to present the umbilicus by a single hand movement such that the arteries or veins are exposed for the insertion of a catheter.

According to a preferred refinement, the hooks can be spring-loaded or spring-guided such that they can be hooked into the umbilical cord and unhooked with greater pressure when the ring element is moved into the proximal position, similar to a ball pen refill. The hooks can be held in the spring such that they can be released in order to avoid the risk of injury.

According to another refinement of the present disclosure the sheath of the medical device comprises a substantially cylindrical shape.

The medical device according to the disclosure can be a disposable product or a reusable and possibly sterilizable device. In the latter alternative, the device can be used more than once as a whole, or individual units of the device can be provided as reusable and disposable elements. As a disposable product or as a reusable product, the device and/or the individual units can also be provided in different sizes.

The entire medical device can be made of plastic and can be produced, for example, by 3D printing or injection moulding. Individual elements or components or the device as a whole can also be made of other materials. Elements such as knives, needles, hooks, and other technical functional components can be made of metal or stainless steel.

Further advantages result from the figures of the following description of preferred embodiments. It is to be understood that the features mentioned above and the features to be explained below can be used not only in the respective combination indicated, but also in other combinations or alone, without leaving the scope of this disclosure. Embodiments of the disclosure are shown in the figures and are explained below.

### BRIEF DESCRIPTION OF THE DRAWINGS

FIG. 1 a schematic drawing of a first embodiment of the first unit of the medical device comprising a clamping device for clamping the umbilical cord;

FIGS. 2A-2C schematic drawings of a first embodiment of the second unit of the medical device comprising a severing device for severing the umbilical cord to form an umbilical cut surface;

FIG. 3 a schematic drawing of a first embodiment of the third unit of the medical device comprising a hooking device, for spreading the umbilical cord cut;

FIG. 4 a schematic drawing of a first embodiment of the medical device for placing a catheter in an umbilical cord blood vessel of a newborn;

FIG. 5 a schematic drawing of a first embodiment of the medical device for placing a catheter in an umbilical cord blood vessel of a newborn, wherein the device being opened; and

FIGS. 6A-6C schematic drawings of the movement mechanism of the third unit of the medical device.

### DETAILED DESCRIPTION

Functionally equivalent elements are identified in all figures with the same reference signs, also in different embodiments.

FIG. 1 shows a schematic drawing of a first embodiment of the first unit 100 of the medical device, which comprises a clamping device 10, for clamping the umbilical cord, in particular the blood vessels.

FIG. 1 shows the first unit 100, which is formed cylindrical. The unit 100 comprises two subunits 14 and 16, which are connected by a hinge 12. In an embodiment not shown, the first unit 100 can be arranged without a hinge 12. For this, the umbilicus is threaded into first unit 100. The hinge 12 offers the advantage that the unit 100 can be placed easy around the umbilicus. The unit 100 or the subunits 14 and 16 are closed by a sheath closing mean 18. This closure 18 can be opened again when required.

The first unit 100 comprises a clamping device 10, which is arranged in the first unit 100. This clamping device 10 is designed for clamping the umbilical cord. According to the disclosure clamping means that the blood vessels, which are located in the umbilicus, are clamped off. This prevents bleeding of the newborn. The clamping device 10 may comprise, for example, a tie 11 or an air cushion. A tie/ribbon 11 can be arranged in the first unit 100 via a loop guide, such that a loop is pulled around the umbilicus by pulling the tie 11, which leads to clamping of the blood vessels. Furthermore, the clamping device 10 can also be designed as an air cushion. The air cushion according to this embodiment is arranged such that the umbilicus of the newborn is clamped by filling the cushion with air. The vessels can be reopened again by the targeted escape of air.

The clamping device 10 is shown in FIG. 1 as a tie 11. This tie 11 is intended to clamp the blood vessels securely, such that bleeding of a newborn is prevented. The clamping device 10 is also designed such that the blood vessels are at least partially reopened when a catheter is placed. Thus, the clamping device 10 can therefore be opened and closed reversibly. The tie 11 is arranged in the first unit 100 such that the umbilicus is clamped by pulling the tie 11. Here, the umbilicus is not ruptured. Furthermore, the clamping device 10 ensures that the clamping device 10 is not released in undesirable ways.

Preferably, the first unit 100 is applied as close as possible to the body of the newborn.

In the shown embodiment the first unit 100 comprises also connecting means 19. These connecting means 19 serves to fasten/connect the first unit 100 to/with the third unit. The connecting means 19 are formed as bayonet lock. The two units can be connected easily and fast by inserting the third unit, which comprises the corresponding bolts, into the others and rotating it in the opposite direction.

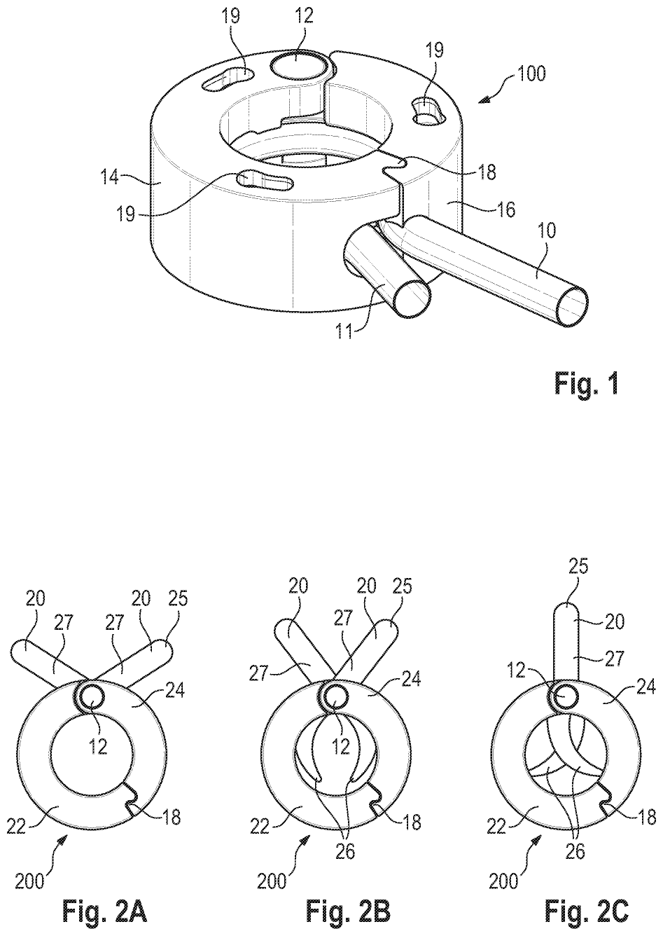

FIG. 2A-2C shows a schematic drawing of the first embodiment of the second unit 200 of the medical device, which comprises a severing device 20 for severing the umbilical cord to form an umbilical cut surface. The figures show in particular the mechanism of the severing device 20.

The second unit 200 is formed cylindrically and comprises two subunits 22 and 24, which are connected by a hinge 12. The severing device 20 is also arranged at the hinge 12, such, that it can be guided over the hinge 12. The subunits 22 and 24 can be locked by a sheath closing mean 18. The sheath closing mean 18 is formed as a simple hooking lock, which can be opened and closed again as required.

The severing device 20 is suitable for severing the umbilical cord to form an umbilical cut surface. The severing device 20 comprises cutting means 25 among other things. In these figures the severing device 20 is shown as scissor-like cutting means 25. Other preferred cutting means 25 are knife-like cutting means or thread-like cutting means. The cutting means 25 can be a scalpel, scissors, metallic thread or the like. The severing device 25 is arranged in the second unit 200 and can be activated by a simple movement mechanism. Preferably, the cutting means 25 are inserted in the second unit 200 such that the umbilicus can be cut by moving the second unit 200 or the cutting means 25 with one hand. Preferably, the cutting surface is smooth such that the umbilical vessels are not frayed. In a particular embodiment, the severing device 20 can be activated by rotating the second unit 200.

The cutting means 25 in FIG. 2A to 2C comprises two or more blades 26 and one lever end 27 each. The blades 26 with their lever ends 27 are movably connected via a hinge 12. The blades 26 are formed curved. This provides the advantage that they can be attached in the cylindrically shaped subunits 22 and 24 and that the umbilicus can be cut preferentially. According to another embodiment, the blades 26 can comprise a different shape (not shown).

The blades 26 can be pushed against each other like scissors such that the umbilical cord can be severed. FIG. 2A shows the lever ends 27 of the knife blades 26. These protrude, while the knife blades 26 in the second unit 200 are attached such that the knife blades 26 cannot cut in this position. The knife blades 26 are also shifted against each other by the opposite guidance of the lever ends 27, such that they can sever the umbilical cord. This is shown in FIGS. 2B and 2C.

The cutting means 25 are preferably arranged at the proximal end of the second unit 200, such that after cutting the umbilicus a part of the umbilicus falls off together with the second unit 200. According to a preferred embodiment the second unit 200 with its proximal end is as close as possible to the distal end of the third unit. According to this embodiment, the spreading of the umbilical cut surface can be carried out particularly advantageously. The further away the second unit 200 and the third unit are from each other, the more difficult it is to spread the umbilical cut surface.

According to a not shown embodiment, the severing device 20 may comprise a cutting mean 25 which is guided along a rail.

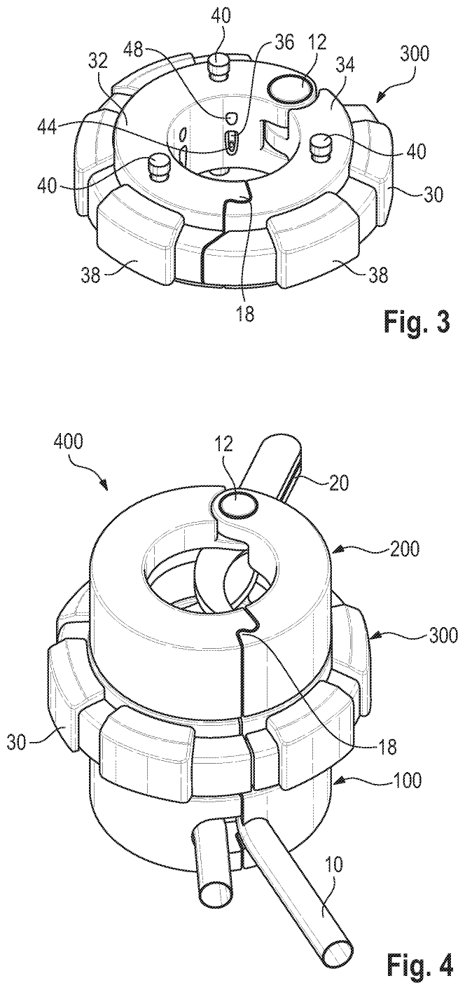

FIG. 3 shows a schematic drawing of a first embodiment of the third unit 300 of the medical device comprising a hooking device 30 for spreading the umbilical cord cut.

The third unit 300 is cylindrical and comprises two subunits 32 and 34, which are connected by a hinge 12. The hooking device 30 is partly arranged around and inside the subunits 32 and 34. The subunits 32 and 34 with the hooking device 30 can be closed by a sheath lock 18. The sheath lock 18 is designed as a simple reclosable hooking lock.

The hooking device 30 is designed such that it is suitable for spreading an umbilical cut surface. The hooking device 30 comprises, among other things, hook elements 36, which are attached to a ring element 38. The ring element 38 is arranged to the cylindrical sheath or to the subunits 32 and 34 of the third unit 300. The hook elements 36 are attached to the ring element 38 at their fastening end 48. The second hook end 44 of the hook elements 36 is arranged on the inside of the sheath. These hook ends 44 are designed such that they can hook into the umbilical tissue.

The third unit 300 also comprises connecting means 40. These connecting means 40 are intent to fasten the third unit 300 to the first unit and/or the second unit. The connecting means 40 are designed as bayonet fasteners, in which case the third unit 300 comprises the bolt. Accordingly, the first or second unit could comprise notches in which the bolts can be inserted and twisted into each other. This allows the units to be connected easy and fast.

FIG. 3 also shows that the hook elements 36 are arranged in a type of guide rail, which allows the ring element 38 to be guided in the distal and proximal longitudinal directions. If the ring element 38 is moved from the distal end to the proximal end, the hook elements 36 hook into the umbilical tissue and spread it open. The mechanism or movement sequence of the spreader is shown schematically in FIGS. 6A to 6C.

FIG. 4 shows a schematic drawing of the medical device 400 for placing a catheter in an umbilical cord blood vessel of a newborn. The umbilical cord blood vessels are two arteries and one vein.

The medical device 400 comprises three units, 100, 200 and 300, which are connected to each other. The connection between the individual units 100, 200 and 300 is reclosable such that the individual units 100, 200 and 300 can be separated from each other and can be reclosed. In particular, the easy separation of the individual units 100, 200 and 300 make this possible. The individual units 100, 200 and 300 can, for example, be separated from each other by rotating them against each other. Other locking units can be, for example, bayonet connections, swivel connections or pull connections. The individual units 100, 200 and 300 are preferably connected to each other such that the lock can be opened or closed with one or two hands.

The first unit 100 is preferably attached to the proximal end of the umbilicus. The second unit 200 is preferably attached at the distal end of the umbilicus, the third unit 300 being attached between the first unit 100 and the second unit 200 closed around the umbilicus. In this embodiment/view, the medical device 400 is shown in a closed position as a cylindrical sheath, which can be opened and closed along the longitudinal axis. According to this embodiment, it is easy for a treating person to place the medical device 400 around the umbilical cord and then securely close/lock it. Preferably, the closure 18 is designed, such, that the cylindrical sheath cannot open unintentionally during treatment. This sheath lock 18 can, for example, be opened or pushed open. For example, the sheath lock 18 can be formed as a clamp, click, hinge or rail means. According to the disclosure, the sheath locking device is a locking device that is particularly easy to lock. Preferably, the sheath can be closed with one hand.

According to an embodiment not shown, the medical device 400 may be configured such that it comprises, in addition to the third unit 300, only the first unit 100 or only the second unit 200. Thus, the medical device 400 would consist of only two units.

The medical device 400 can be available in different sizes. Preferably, the inner diameter of the medical device corresponds about to the umbilicus diameter of the newborn.

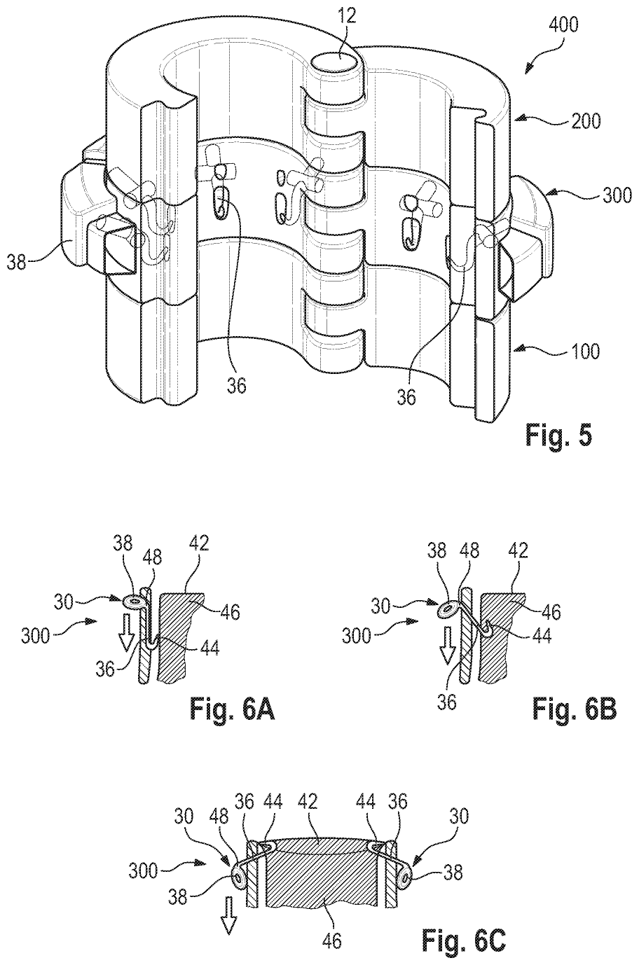

FIG. 5 shows a schematic drawing of the first embodiment of the medical device 400 for placing a catheter in an umbilical cord blood vessel of a newborn, with the device 400 being in an open position.

The first, second and third units 100, 200 and 300 of the medical device 400 are shown. The individual units 100, 200 and 300 are connected to each other. For sake of clarity, units 100 and 200 are shown to not comprise their clamping- or severing devices in this figure. Otherwise, the depicted medical device 400 corresponds to the one in FIG. 4. In particular, the hook elements 36 can be seen in this figure, which are attached on the ring element 38 or in the third unit 300.

FIG. 6A-6C show a schematic drawing of the movement mechanism or the movement sequence of the third unit 300 of the medical device 400, which leads to the spreading of the umbilical cut surface 42. This is achieved by a sequence of movements of the hooking device 30 of the third unit 300.

FIG. 6A shows the cylindrical sheath of the third unit 300 in which the hooking device 30 is provided. The hooking device 30 comprises, among other things, hook elements 36. This hook element 36 comprises a first fastening end 48 and a second hook end 44. Via the first fastening end 48 the hook elements 36 are fastened to a ring element 38. This ring element 38 is arranged circumferentially on the cylindrical sheath. The second hooking end 44 is designed such that it can hook into the umbilical cord tissue 46.

By moving the ring element 38 in the proximal longitudinal direction, as shown in FIG. 6B, the second hooking end 44 gets hooked into the umbilical cord tissue 46. By moving the ring element 38 further in the proximal direction, the umbilical cut surface 42 is spread out, as shown in FIG. 6C.

It is to be understood that the foregoing description is of one or more preferred example embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

What is claimed is:

1. A medical device for placing a catheter in an umbilical cord blood vessel of a newborn, wherein the medical device comprises a longitudinal axis, a proximal and a distal end, and being sheath-like designed to enclose a portion of an umbilical cord, wherein the medical device comprises the following:
a first unit, comprising a clamping device for clamping the umbilical cord, and/or
a second unit, comprising a severing device for severing the umbilical cord to form an umbilical cut surface, and
a third unit, comprising a hooking device for spreading the umbilical cut surface.

2. The medical device according to claim 1, wherein the first unit is arranged at the proximal end of the device and the second unit is arranged at the distal end of the device, wherein the third unit is arranged between the first and the second unit of the device.

3. The medical device according to claim 1, wherein the first, second and third units are connectable to one another via connecting elements, such, that the first unit is attachable in the device by rotation against the second and/or the third unit.

4. The medical device according to claim 1, wherein the first, second and third units are connectable to one another via connecting means, such that the first unit is attachable in the device by rotation against the second and/or the third unit, wherein the connecting elements are selected from rotational connection elements, bayonet connection elements, rail connection elements, hook connection elements and/or magnet connection elements.

5. The medical device according to claim 1, wherein the sheath is closed for threading the umbilical cord into the sheath.

6. The medical device according to claim 1, wherein the sheath is designed such, that it can be opened or slid open or pushed open along the longitudinal axis of the device and can be locked again to close the sheath for placing the device around a section of the umbilical cord.

7. The medical device according to claim 1, wherein the sheath can be opened or slid open or pushed open along the longitudinal axis of the device and can be locked again to close the sheath for placing the device around a section of the umbilical cord, wherein the first, second and third unit each comprises sheath closing elements for opening or slide open the sheath, wherein the sheath closing elements are selected from clamp, click, hinge, hook, and rail elements.

8. The medical device according to claim 1, wherein the clamping device comprises a tie or an air cushion for clamping the blood vessels in the umbilical cord at the proximal end of the medical device.

9. The medical device according to claim 1, wherein the clamping device is designed such that the umbilical cord is clamped by equatorial rotation of the first unit against the second and/or third unit.

10. The medical device according claim 1, wherein the severing device comprises cutting elements.

11. The medical device according to claim 1, wherein the severing device comprises cutting elements selected from a knife-like cutting element, a scissor-like cutting element or a thread-like cutting element.

12. The medical device according to claim 1, wherein the severing device comprises a cutting element selected from a knife-like cutting element, wherein the knife-like cutting element is movably attached to a rail or the knife-like cutting element is movably attached to a hinge or the knife-like cutting element is spring-loaded.

13. The medical device according to claim 1, wherein the severing device of the second unit is designed, such, that the umbilical cord is clamped by equatorial rotation of the second unit against the first and/or the third unit, and wherein the second unit is detachably arranged on the device.

14. The medical device according to claim 1, wherein the hooking device comprises at least three hook elements.

15. The medical device according to claim 1, wherein the hooking device comprises at least four, five, six, seven, eight, nine or ten hook elements each having a first fastening end and a second hooking end.

16. The medical device according to claim 1, wherein the hooking device comprises hooking elements and a ring element, wherein the hooking elements are each attached to the ring element via a first fastening end, which is attached to the hooking device circumferentially in hinges around the sheath, and wherein a second hooking end of the hooking elements is designed for getting hooked into the umbilical cord tissue.

17. The medical device according to claim 1, wherein the hooking device comprises hooking elements and a ring element, wherein the hooking elements are each attached to the ring element via a first fastening end, which is attached to the hooking device circumferentially in hinges around the sheath, and wherein a second hooking end of the hooking elements is designed for getting hooked into the umbilical cord tissue, wherein the ring element is in distal and proximal longitudinal direction of the medical device is slidably arranged on the hooking device such that by moving the ring element from a distal position to a proximal position the hook elements get hooked in the umbilical cord and can spread the umbilical cut surface.

18. The medical device according to any of claim 1, wherein the sheath comprises a substantially cylindrical shape.

19. A method for placing a catheter in an umbilical cord blood vessel of a newborn using the medical device according to claim 1, comprising the following steps:
placing the medical device according to claim 1 around the umbilical cord of a newborn;
actuating the first unit to effect clamping of blood vessels of the umbilical cord at the proximal end of the device;
actuating the second unit comprising the severing device to effect cutting the umbilical cord and to form an umbilical cut surface;
removing the second unit from the device;
actuating the third unit to effect spreading of the umbilical cut surface; and
placing the catheter in an umbilical cord blood vessel.
